(19) United States
(12) Patent Application Publication
(10) Pub. No.: US 2026/0035642 A1
(43) Pub. Date: Feb. 5, 2026

(54) SYSTEM AND METHOD FOR PRECISE FABRICATION OF BIOMATERIAL-ENCAPSULATED CELL MASSES

(71) Applicant: BIOARCHITEC GROUP LIMITED, Hong Kong (CN)

(72) Inventors: Chung Wai Joanthan LAM, Hong Kong (CN); Chi Hang WONG, Hong Kong (CN); Chi Hang WONG, Hong Kong (CN); Chiu Chin PONG, Hong Kong (CN)

(21) Appl. No.: 18/997,870

(22) PCT Filed: Jul. 25, 2023
(86) PCT No.: PCT/CN2023/109057

§ 371 Date: Jan. 23, 2025

Related U.S. Application Data
(60) Provisional application No. 63/369,256, filed on Jul. 25, 2022.

(51) Int. Cl. C12M 3/00; C12M 1/00; C12M 1/26; C12M 1/34; C12N 5/071

(52) U.S. Cl. CPC C12M 21/08; C12M 23/34; C12M 29/06; C12M 33/14; C12M 41/46; C12N 5/0697; C12N 2513/00; C12N 2537/10

(57) ABSTRACT

The present invention provides a system for performing high-throughput selection of cell masses from a source, fabrication of biomaterial-encapsulated cell mass model based on the selected cell masses, and separation of biomaterial-encapsulated cell mass model, cell masses or cells from a pool of biomaterial-encapsulated cell mass models as-fabricated in a fully automated manner to minimize selection errors due to human intervention and potential contaminations to biological samples during liquid handling among different analytical devices or units.

### CROSS-REFERENCE TO RELATED APPLICATION

This application claims priorities from the U.S. provisional patent application Ser. No. 63/369,256 filed Jul. 25, 2022, the disclosure of which is incorporated herein by reference in its entirety.

### TECHNICAL FIELD

The present invention relates to a system for high-throughput selection, fabrication, and separation of biomaterial-encapsulated cell mass (BECM), and related method for producing the BECM.

### BACKGROUND

Cell masses, including single-cell type spheroids and multi-cell type organoids derived from any living organisms, are important models for in vitro and in vivo studies such as cancer research, drug screening, and drug sensitivity test for precision medicine. However, current technologies result in inconsistent cell number and sizes during the production of these cell masses. In the context of cancer research, the composition of normal and cancerous content in patient-derived organoids (PDO) is highly inconsistent among individual cell masses. The tissue source for PDO culturing taken from the patient is usually limited in size, which also limits the number of organoid cell masses that can be produced, and therefore the subsequent tests that can be performed. This hinders their applications in drug test and development with the recent advancement in precision medicine.

Due to the inherent tumor cell diversity, researchers usually derive cell masses from primary cells after careful selection, but current technologies hinder their selective power and control over the size, composition, and cell number of the cell masses such as spheroids and organoids derived from tissues or biopsy of a donor. In case of multi-cell type organoids, a mixture of highly variable compositions and sizes of the cell masses inherently imposes inconsistency to experimental results, which are usually compensated by increasing the sample size. To allow subsequent tests, the multi-cell type organoids should be digested into single cells before seeding equal number of cells in each well for accurate comparisons. This involves an assumption that all the cell compositions are equally divided and the multi-cell type organoids grown from the single cells are formed at the same rate with similar compositions. However, each individual cell mass is in fact different from the other, so the assumption as such leads to an even more inconsistent result.

Cell sorting by flow cytometry or cell sorter could be a solution to attempt to standardize the cell size or cell stage by biomarkers, but it involves time and labors, and treatment of the cells before sorting may cause other unexpected result to the cell fate and its ability to grow into a desired biological model or cell lineage for various tests.

In a co-pending PCT application under the application number PCT/CN2023/095216 filed on May 19, 2023, which disclosure is also incorporated herein by reference, a method for selection and fabrication of a biomaterial-encapsulated cell mass (BECM) model was provided. A corresponding system to enable a precise, fully automated and substantially contamination-free fabrication method of the BECM is therefore needed.

### SUMMARY OF INVENTION

Accordingly, a first aspect of the present invention provides a system for high-throughput selection of target cell masses, fabrication of biomaterial-encapsulated cell mass (BECM) model under a controlled manner in terms of the number of cell mass, dimension, composition, and number of cells per mass unit with consistency (repeatability), and separation of the as-fabricated BECM model from other cell masses or cell debris. The isolated BECM model, target cells or cell masses derived therefrom are ready for use in a wide range of applications without further cultivation or pre-conditioning.

In the first aspect, the system comprises:
a fabrication chamber comprising at least one fluid inlet, one fluid outlet, and a main channel region for receiving and cultivating cell masses and fabricating the BECMs from the cell masses;
a multi-axial movable mechanism disposed adjacent to, above or under the fabrication chamber for controlling a multi-axial movement of the fabrication chamber;
a photo-stimulation unit disposed above or under the fabrication chamber for photocuring the biomaterial to encapsulate target cell masses;
a microscopic device disposed on an opposing side of the photo-stimulation unit for capturing images of the fabrication chamber;
a cell sorting device comprising at least one fluid inlet communicating with the fluid outlet of the fabrication chamber for receiving the fabricated BECMs, a target cell masses detection unit for separating the BECMs from non-target cell masses, and one fluid outlet for outputting a stream of separated BECMs;
a cell model dispenser comprising one end communicating with the fluid outlet of the cell sorting device, and one or more dispenser heads for dispensing the stream of the BECMs separated by the cell sorting device; and
a receptable for receiving the separated BECMs from the cell model dispenser.

In certain embodiments, the cell masses are sourced from primary, secondary or modified cell lines, or a tissue or biopsy of the same or different subject than a recipient of the BECM or the cells derived therefrom.

In certain embodiments, the cell masses include spheroids and organoids.

In certain embodiments, the fabrication chamber is a microfluidics, a single-channel device, or a multi-channel device.

In certain embodiments, the fabrication chamber is optically transparent.

In certain embodiments, the main channel region of the fabrication chamber comprises multiple compartments.

In certain embodiment, each of the compartments may comprise a temperature control mechanism and a gas regulation mechanism to maintain cell cultivation conditions for the cell masses.

In certain embodiments, the microscopic device comprises a microscope and a camera.

In certain embodiments, the microscope comprises a light source comprising incandescent tungsten-halogen bulb, laser or light emitting diode (LED).

In certain embodiments, the light source of the microscope has a wavelength from 488 to 633 nm.

In certain embodiments, the microscope can be adjusted with the depth of focus.

In certain embodiments, the microscopic device is configured to detect optical signals including visible light, fluorescent, luminescent, scattering, absorbance, and turbidity signals and output bright-field, dark-field, fluorescent, and luminescent images.

In certain embodiments, all the cell masses are pre-labelled with one or more fluorescent or luminescent probes specific to one or more biomarkers, and the optical signals detectable from the cell masses represent expression of the one or more biomarkers corresponding to one or more target cell masses to be encapsulated in the biomaterial.

In certain embodiments, the system further comprises an image analysis software for processing and analyzing the images captured by the microscopic device.

In certain embodiments, the target cell masses will be mapped in the corresponding compartment(s) based on region of interests (ROIs) identified in the images by the image analysis software in order to determine three-dimensional (3-D) coordinates of the target cell masses in the fabrication chamber.

In certain embodiments, the image analysis software is configured to differentiate the optical signals from the target cell masses than those from the non-target cell masses or any noise from the background, and also calculate distance between the target cell masses and their neighboring non-target cell masses.

In certain embodiments, the target cell masses have an average diameter from about 30 to 2000 μm.

In certain embodiments, the target cell masses have an average diameter from about 80 to 100 μm.

In certain embodiments, each of the target cell masses and its neighboring non-target cell masses are separated by a distance from larger than 0 μm to 2000 μm.

In certain embodiments, the distance between any one of the target cell masses and its neighboring non-target cell masses is from at least 200 μm to 2000 μm.

In certain embodiments, the non-target cell masses have an average diameter larger than 20 μm.

In certain embodiments, the photo-stimulation unit comprises at least one light source with various wavelengths and a plurality of filters.

In certain embodiments, the light source of the photo-stimulation unit has a wavelength of about 405 nm and the plurality of filters is long-pass filters.

In certain embodiments, the multi-axial movable mechanism is a three-dimensional (3-D) translational stage capable of moving along at least two axes, e.g., along x- and y-axes which are perpendicular to the light source direction of the microscopic device, respectively.

In certain embodiments, the 3-D translational stage is disposed under the fabrication chamber.

In certain embodiments, the photo-stimulation unit is disposed under the fabrication chamber and within the 3-D translational stage.

In certain embodiments, the multi-axial movable mechanism is a movable clamping structure for securing the fabrication chamber to the multi-axial movable mechanism and moving the fabrication chamber along at least two axes perpendicular to the light source direction of the microscopic device, respectively; the photo-stimulation unit can be disposed under or above the fabrication chamber.

In certain embodiments, one or more compartments of the main channel region of the fabrication chamber are masked by the plurality of filters of the photo-stimulation unit such that the light source of the photo-stimulation unit only reaches the unmasked compartments.

In certain embodiments, masking of the one or more compartments by the plurality of filters is controlled by the image analysis software.

In certain embodiments, the image analysis software is also configured to control the movement of the multi-axial movable mechanism according to the 3-D coordinates of the target cell masses in the fabrication chamber and align with the light path of the light source of the photo-stimulation unit.

In certain embodiments, the target cell masses detection unit of the cell sorting device comprises a quenching mechanism comprising a light scattering device.

In certain embodiments, the target cell masses detection unit is controlled by the image analysis software to automatically identify the BECMs and separate the same from the non-target cell masses.

In certain embodiments, the target cell masses detection unit will identify the BECMs based on a set of selection criteria determined by the image analysis software or pre-determined by the user.

In certain embodiments, the cell sorting device and the cell model dispenser are configured into a single unit.

In other embodiments, the cell sorting device and the cell model dispenser are two separate units.

In certain embodiments, the cell model dispenser further comprises a detection unit comprising ultrasound object detector, infrared detector photomultiplier tubes (PMT), photodiodes and excitation light sources.

In certain embodiments, the receptacle comprises multi-well plates, petri dishes, centrifuge tubes or other common labware capable of receiving and carrying separated BECMs from the cell model dispenser.

In certain embodiments, the receptacle should be sterile and pathogen-free in order to minimize contaminations by the external environment.

In certain embodiments, the fabrication chamber, the cell sorting device, the fluid channel communicating the fabrication chamber and the cell sorting device, the cell model dispenser and the receptacle are configured to be isolated from the external environment and be kept in a substantially sterile and pathogen-free condition. In other words, all the biomaterials, cell masses, and reagents used or produced in selection of target cell masses, precise fabrication of BECMs and separation thereof from the rest of the non-target cell masses or cell debris among the fabrication chamber, the cell sorting device, the fluid channel, the cell model dispenser and the receptacle are sealed from the external environment to avoid contamination of the biological samples and the as-fabricated BECMs by external factors such as pathogens.

A second aspect of the present invention provides a method for fabricating biomaterial-encapsulated cell mass model comprising using the system according to the first aspect. The method comprises:
providing a pool of cell masses;
pre-labelling the cell masses with one or more fluorescent or luminescent probes specific to one or more biomarkers expressed in target cell masses;
mixing the pre-labelled cell masses with a biomaterial formulation to form a first mixture, wherein the biomaterial formulation comprises a precursor of the biomaterial;
capturing a first batch of images of the fabrication chamber for constructing a three-dimensional geometry of the fabrication chamber;
providing the first mixture to the fabrication chamber;
capturing a second batch of images of the fabrication chamber provided with the first mixture to detect any fluorescent or luminescent signals;
determining region of interests (ROIs) from the second batch of images;
identifying centroids of the cell masses from the ROIs meeting a set of threshold values with the presence of fluorescent or luminescent signals;
determining three-dimensional (3-D) coordinates of the target cell masses in the fabrication chamber;
selectively encapsulating the target cell masses into the biomaterial according to the determined three-dimensional coordinates of the target cell masses in the fabrication chamber in order to form a second mixture comprising biomaterial-encapsulated cell masses (BECMs);
transferring the second mixture to the cell sorting device for separation of the BECMs from non-target cell masses or cell debris; and
dispensing the separated BECMs to the receptacle through the cell model dispenser.

In certain embodiments, the pool of cell masses is sourced from primary, secondary or modified cell lines, or a tissue or biopsy of the same or different subject than a recipient of the BECM or the cells derived therefrom.

In certain embodiments, the cell masses include spheroids and organoids.

In certain embodiments, the fabrication chamber is a microfluidics, a single-channel device, or a multi-channel device.

In certain embodiments, the microfluidics comprises microfluidic chip, microfluidic disc, and microfluidic cell.

In certain embodiments, the fabrication chamber comprises at least one fluid inlet, one fluid outlet, and a main channel region.

In certain embodiments, the fabrication chamber is optically transparent, and can be made of glass, plastic, or any composite material thereof.

In certain embodiments, the first mixture is provided through the fluid inlet to the fabrication chamber.

In certain embodiments, the main channel region of the fabrication chamber comprises multiple compartments.

In certain embodiments, the precursor of the biomaterial can form a network, gelation system, or scaffold for encapsulating and accommodating the target cell masses to form the BECMs in the fabrication chamber upon stimulation by one or more elements comprising a light irradiation, a change of pH or temperature, or a change in chemical composition of the biomaterial formulation.

In certain embodiments, the precursor of the biomaterial is a photo-crosslinkable hydrogel.

In certain embodiments, the photo-crosslinkable hydrogel is gelatin-methacryloyl (Gel-MA), alginate-methacryloyl, hyaluronic acid-methacryloyl, fibroin-methacyloyl, chitosan-methacryloyl, poly(ethylene glycol)-methacryloyl, dextran-methacryloyl, poly-lysine-methacryloyl, or F127-methacryloyl, or any combination thereof.

In certain embodiments, the photo-crosslinkable hydrogel can be a mixture of more than one of acrylated polymers, and the ratio between different acrylated polymers in the mixture varies according to the desired viscosity of the mixture and/or strength of the resulting biomaterial after crosslinking.

In certain embodiments, the biomaterial formulation further comprises a photo-initiator.

In certain embodiments, the photo-initiator is lithium phenyl-2,4,6-trimethylbenzoylphosphinate (LAP), 2-hydroxy-4′-(2-hydroxyethoxy)-2-methylpropiophenone, or 2,4,6-trimethylbenzoyldi-phenylphosphinate, or any combination thereof.

In certain embodiments, the biomaterial formulation is pre-warmed to 37° C. prior to mixing with the cell masses.

In certain embodiments, the biomaterial formulation is induced to form the network, gelation system, or scaffold by a stimulation selectively applied to the fabrication chamber at where the three-dimensional coordinates of the target cell masses are determined.

In certain embodiments, the photo-crosslinkable hydrogel in the presence of the photo-initiator is cross-linked under a selective light irradiation by a photo-stimulation unit to the fabrication chamber at where the three-dimensional coordinates of the target cell masses are determined.

In certain embodiments, the photo-stimulation unit comprising at least one light source and a plurality of filters.

In certain embodiments, the light source of the photo-stimulation unit is configured to generate light beams and the plurality of filters is a plurality of long-pass filters.

In certain embodiments, the light beams have a wavelength of 405 nm and a diameter of about 400 μm to selectively irradiate at the determined three-dimensional coordinates of the target cell masses in the fabrication chamber to form the biomaterial-encapsulated cell masses.

In certain embodiments, the plurality of filters is used to mask one or more of the compartments of the main channel region of the fabrication chamber such that the light source of the photo-stimulation unit only reaches the unmasked compartments.

In certain embodiments, masking of the one or more compartments by the plurality of filters is controlled by the image analysis software according to the 3-D coordinates of the target cell masses.

In certain embodiments, a multi-axial movable mechanism attached to the fabrication chamber for actuating the fabrication chamber along at least two axes that are perpendicular to the light source direction of the microscopic device, respectively is also controlled by the image analysis software.

In certain embodiments, the multi-axial movable mechanism is a 3-D translational stage disposed under the fabrication chamber.

In certain embodiments, the photo-stimulation unit is disposed under the fabrication chamber and within the 3-D translational stage.

In certain embodiments, the multi-axial movable mechanism is a movable clamping structure for securing the fabrication chamber to the multi-axial movable mechanism and actuating the fabrication chamber along the at least two axes perpendicular to the light source direction of the microscopic device, respectively, wherein the photo-stimulation unit can be disposed under or above the fabrication chamber.

Apart from photocuring by light irradiation, the encapsulation of target cell masses into the biomaterial may be performed by one of the following methods: Fused Deposition Modeling (FDM), Selective Laser Sintering (SLS), Digital Light Processing (DLP), Stereolithography (SLA), Single or Multiple photon polymerization, laser writing technology, Laser gelation methods, Droplet-On-Demand (DOD), droplet emulsification and physical cropping.

In certain embodiments, the first and second batches of images are captured by a microscopic device comprising a microscope and a camera.

In certain embodiments, the microscope comprises a light source comprising incandescent tungsten-halogen bulb, laser or light emitting diode (LED).

In certain embodiments, the microscope can be adjusted with the depth of focus.

In certain embodiments, the microscopic device is configured to detect optical signals including visible light, fluorescent, luminescent, scattering, absorbance, and turbidity signals and output bright-field, dark-field, fluorescent, and luminescent images.

In certain embodiments, said constructing the three-dimensional geometry of the fabrication chamber from the first batch of images, said determining the ROIs from the second batch of images and identifying centroids of the cell masses from the ROIs with the presence of fluorescent or luminescent signals, and said determining the 3-D coordinates of the target cell masses in the fabrication chamber are all performed by the image analysis software.

In certain embodiments, the image analysis software is also configured to differentiate the optical signals from the target cell masses than those from the non-target cell masses or any noise from the background, and also calculate distance between the target cell masses and their neighboring non-target cell masses.

In certain embodiments, each of the target cell masses and its neighboring non-target cell masses should be separated by a distance from larger than 0 μm to 2000 μm in the fabrication chamber such that the set of selection criteria is met.

In certain embodiments, the distance between any one of the target cell masses and its neighboring non-target cell masses is set to be from at least 200 μm to 2000 μm in the fabrication chamber.

In certain embodiments, the separation of the BECMs from the non-target cell masses or cell debris in the cell sorting device is performed by the target cell masses detection unit, and the target cell masses detection unit is controlled either by the image analysis software or by a master software that allows the target cell masses detection unit to automatically identify the BECMs based on the set of selection criteria and separate them from the non-target cell masses or cell debris in the second mixture.

In certain embodiments, the set of selection criteria comprises one or more of average diameter of the target cell masses, average diameter of non-target cell masses, expression of one or more biomarkers, composition of BECMs, surface uniformity, content consistency, fluorescence intensity upon quenching, etc.

In certain embodiments, the target cell masses have an average diameter from about 30 to 2000 μm.

In certain embodiments, the target cell masses have an average diameter from about 80 to 100 μm.

In certain embodiments, the non-target cell masses have an average diameter larger than 20 μm.

In certain embodiments, the target cell masses detection unit of the cell sorting device comprises a quenching mechanism comprising a light scattering device.

In certain embodiments, the cell sorting device and the cell model dispenser are configured into a single unit.

In other embodiments, the cell sorting device and the cell model dispenser are two separate units.

In certain embodiments, the cell model dispenser further comprises a detection unit comprising ultrasound object detector, infrared detector photomultiplier tubes (PMT), photodiodes and excitation light sources.

In certain embodiments, the receptacle comprises multi-well plates, petri dishes, centrifuge tubes or other common labware capable of receiving and carrying separated BECMs from the cell model dispenser.

In certain embodiments, the receptacle should be sterile and pathogen-free in order to minimize contaminations by the external environment.

In certain embodiments, the method further comprises removing any fluid containing non-target cell masses, cell debris and other materials from the fabrication chamber, cell sorting device, and cell model dispenser by one or more rounds of buffer washing prior to providing a subsequent mixture of the cell masses and the biomaterial formulation to the fabrication chamber of the present system.

In certain embodiments, the buffer for buffer washing may be pre-warmed before being provided to one of the at least one fluid inlet of the fabrication chamber.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 schematically depicts a cross-sectional view of the present system according to a first embodiment;

FIG. 2 schematically depicts a method of selection, fabrication, and separation of BECMs using the present system depicted in FIG. 1 according to the first embodiment;

FIG. 3 schematically depicts a cross-sectional view of the present system according to a second embodiment;

FIG. 4 schematically depicts a cross-sectional view of the present system according to a third embodiment and a related method of selection, fabrication and separation of BECMs using the same;

FIG. 5 schematically depicts a fabrication chamber of the present system according to certain embodiments;

FIG. 6 is a flow chart showing steps of selective bio-fabrication of BECMs and sorting of as-fabricated BECMs for subsequent high-throughput analysis according to certain embodiments.

FIG. 7 shows image analysis results for determining positions, size and composition of selected spheroids according to certain embodiments: (a) merged fluorescence images generated from the camera; (b) the area with colocalized green and red fluorescence signals; (c) the image analysis algorithm-generated binary image showing the position of the spheroids selected from (b) with desired size and composition, in which green fluorescence is indicated by upwards arrow while red fluorescence is indicated by downwards arrow;

FIG. 8 shows a top view of compartments containing spheroids and biomaterial for encapsulation of selected spheroids according to certain embodiments, where the circular spots denote the irradiation area of 400 μm and the center of each spot is the selected spheroid.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The following examples are provided to assist the understanding of enabling the present invention and should not be considered limiting the scope of the present invention. The scope of the present invention should be referred to the appended claims.

### Example 1—Structure of Present System and Basic Mechanisms for Selection, Fabrication and Separation of Cell Models Based on the Same

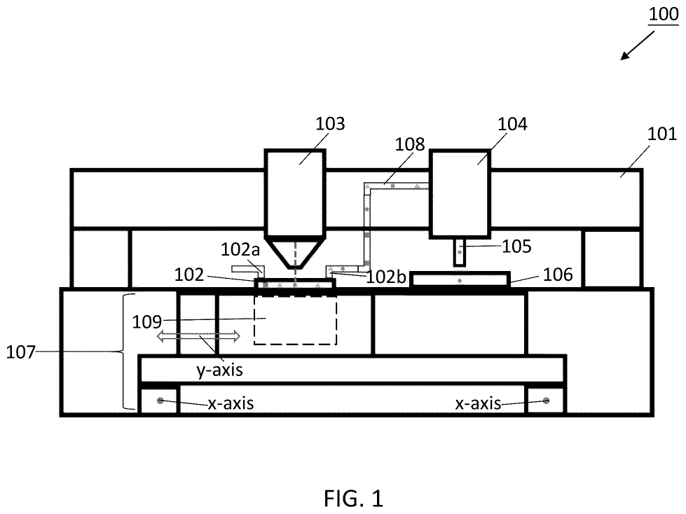

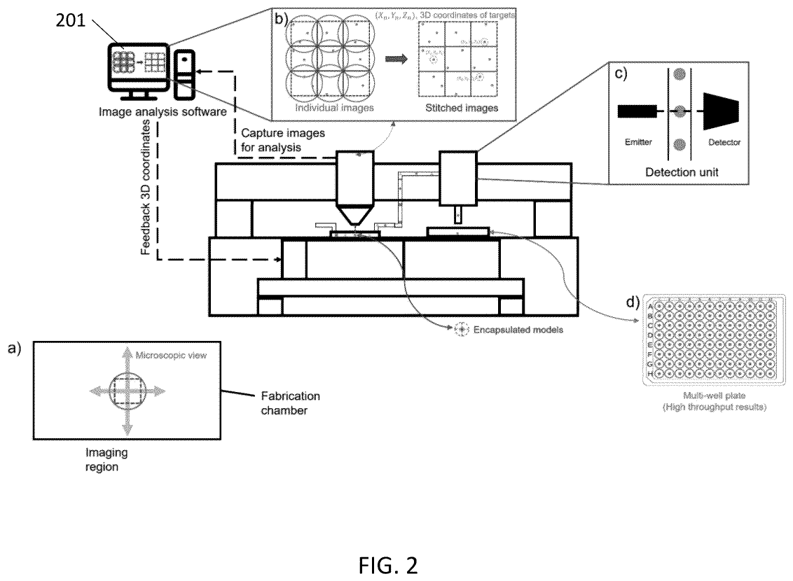

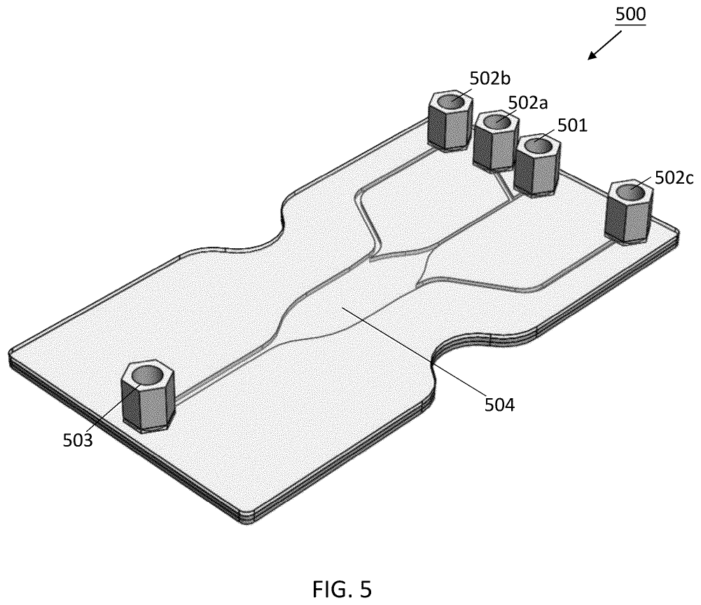

FIG. 1 illustrates a first embodiment of the present system 100 for selection, fabrication, and separation of BECMs, which include a housing 101, a fabrication chamber 102, a microscopic device 103, a cell sorting device 104, a cell model dispenser 105, a receptacle 106, and a three-dimensional (3-D) translational stage 107. The fabrication chamber 102 includes a fluid inlet 102a, a main channel region 102b, and a fluid inlet 102c. As seen in FIG. 2b, the main channel region 102b may be divided into multiple compartments. In certain embodiments, the fabrication chamber 102 is a multi-channel fluid device. More specifically, the fabrication chamber 102 is a microfluidics such as microfluidic chip and microfluidic disc. The fabrication chamber 102 can also be a closed container carrying a mixture of cell masses and a biomaterial for encapsulation of the cell masses. In the case of microfluidic chip, such as the embodiments depicted in FIG. 5, there is also provided with an exit port 503 on the chip to collect the encapsulated cell masses from the mixture. The microfluidic chip may also include, but not limited to, microfluidic valves, microchannels, fluids mixing chamber, filtration membrane, collection chamber and waste chamber. For handling a large amount of biomaterials and cell mass samples, the system may also be configured to pump biomaterials, cell masses, buffer or other liquids into or out of the fabrication chamber in a repetitive manner to achieve a high throughput selective bio-fabrication. It is preferable to have the present system been configured into a substantially sterile manner to avoid any contaminations caused by external factors such as transferring biological materials from one unit to the other of the system in an opened, non-sterilized environment.

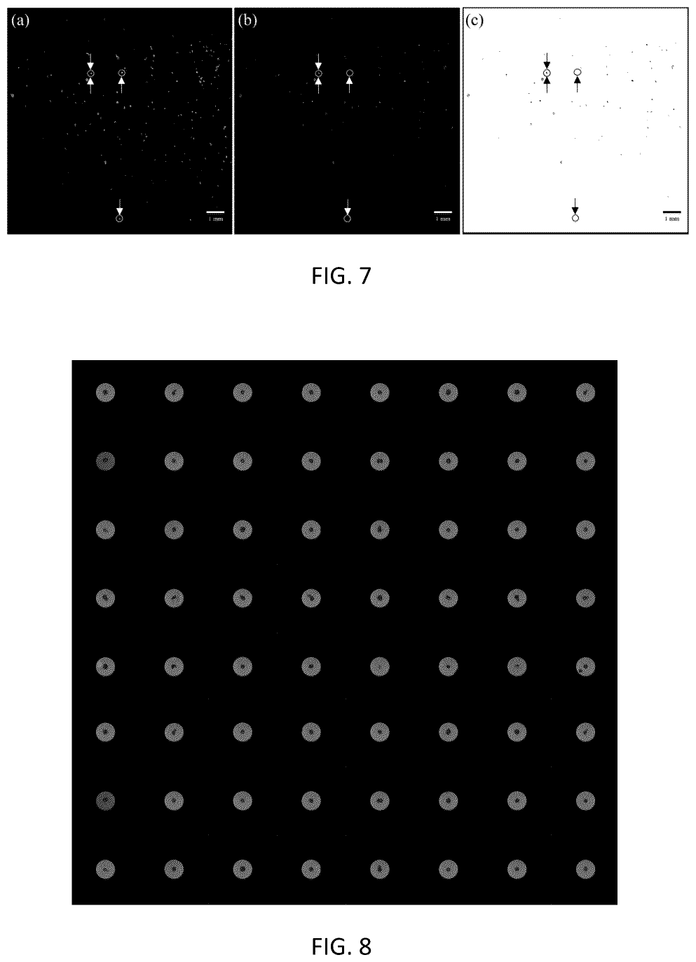

Accordingly, the fabrication chamber 102 according to the first embodiment is connected to the cell sorting device 104 through a fluid connection 108. The fabrication chamber 102 is attached to the 3-D translational stage 107 such that the fabrication chamber 102 could be actuated according to the movement of the 3-D translational stage 107. To achieve precise and fully automated positioning of the fabrication chamber to a corresponding photo-stimulation source (e.g., a photo-stimulation source 109 embedded into the 3-D translational stage 107 as depicted in FIG. 2), the movement of the 3-D translational stage 107 is controlled by an image analysis software 201 which is configured to process and analyze the image data captured by the microscopic device 103 and identify target cell masses from a pool of cell masses in the fabrication chamber according to the image data. The image analysis software identifies the region of interests (ROIs) and selects the interested cell spheroids based on the selection criteria. These selection criteria may include, but not limited to, mass, dimensions, positions, compositions, surface uniformity, content consistency, extent of labelling signals and adjacent distance between targets. These selection criteria may be changed according to the user's preference and/or learned from internal or external dataset. When certain cell spheroids meet the corresponding selection criteria, they will be indicated as target cell masses to be encapsulated into the biomaterial. Multiple images covering the x-y planar area of the fabrication chamber and all the ROIs could be stitched by a corresponding module of the image analysis software. These images include one or more of bright-field, dark-field, and fluorescent images. An example of fluorescent and dark-field images for use to analyze and determine the ROIs is depicted in FIG. 7. A 3-D geometry of the fabrication chamber and corresponding spatial distribution of the target cell masses in the ROIs can then be generated. The spatial distribution of target cell masses can then be mapped with the 3-D geometry of the fabrication chamber in order to determine their respective 3-D coordinates in the fabrication chamber. Based on these 3-D coordinates, the 3-D translational stage 107 could move along x- and/or y-axes (FIG. 2a) in order to actuate the fabrication chamber 102 attached thereto such that the target cell masses could be exposed to the photo-stimulation source for subsequent photo-curing. Other regions of the fabrication chamber will be masked, e.g., by using some long-pass filters to filter certain regions of light source from the photo-stimulation source, such that the biomaterial in those regions in the fabrication chamber will not be photo-cured in order to achieve selective and precise fabrication of biomaterial-encapsulated cell masses.

Alternatively, the 3-D translational stage may be substituted with a movable clamping structure to secure the fabrication chamber and move the fabrication chamber along x- and y-axes with respect to the light beam path of the light source from the microscopic device during image capturing process or the light beam path of the light source from the photo-stimulation unit during 3-D precise fabrication process of BECMs.

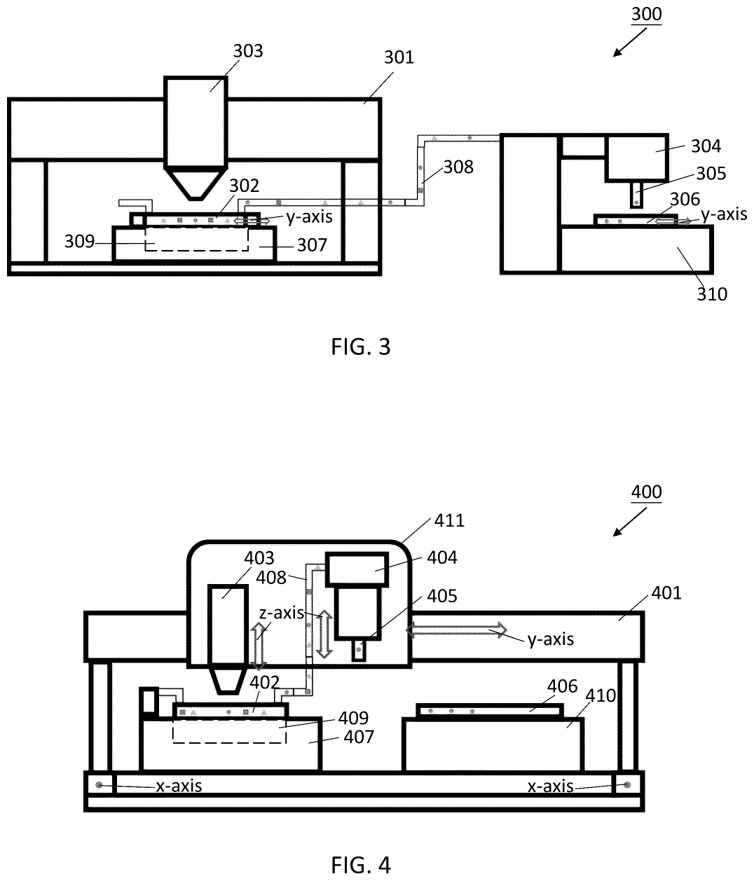

Turning to FIG. 3, a second embodiment of the present system 300 is schematically depicted. Different from the first embodiment depicted in FIG. 1, the second embodiment depicted in FIG. 3 shows that the cell sorting device 304, the cell model dispenser 305, and the receptacle 306 are not integrated into the same housing 301 for accommodating the fabrication chamber 302, the microscopic device 303 and the photo-stimulation source 309 disposed under the fabrication chamber 302, but only communicated with the fabrication chamber 302 through a fluid channel 308. In addition, the fabrication chamber 302 and the receptacle 306 are independently moved along y-axis direction by two separate 3-D translational stages (307, 310) attached thereto, respectively. The two 3-D translational stages (370, 310) are not necessarily disposed at the same horizontal level as seen from FIG. 3. That is, the fabrication chamber as shown on the left hand side can be disposed higher or lower than the receptacle on the right hand side, or vice versa. The arrangement in the second embodiment provides flexibility for further enhancing the sampling size and/or cell sorting speed if needed. Such arrangement also eases replacement or repairment of either or both sections of precise fabrication of BECMs and separation-dispensation of fabricated BECMs from the rest in the sampling fluid.

FIG. 4 depicts a third embodiment of the present system 400, in which partially the microscopic device 403, the cell sorting device 404, the cell model dispenser 405, and partially the fluid channel 408 are accommodated in a semi-opened case 411 being disposed above the fabrication chamber 402 and the receptacle 406. Different from the first and second embodiments depicted in FIG. 1 and FIG. 3, respectively, the microscopic device 403, the cell sorting device 404, and the cell model dispenser 405 are configured to be independently or jointly movable along one-, two-, or even three-axis directions, subject to the needs of the user and the samples to be handled. As seen from FIG. 4, the semi-opened case 411 is configured to be movable along y-axis direction, while the microscopic device 403 and the cell sorting device 404 together with the cell model dispenser 405 are independently movable along z-axis direction, i.e., the vertical position of the microscopic device 403 and the cell sorting device 404/the cell model dispenser 405 is adjustable in this embodiment. Similar to the second embodiment depicted in FIG. 3, the fabrication chamber 402 and the receptacle 406 in the third embodiment are attached with two separate translational stage (407, 410), respectively, in which the translational stage 407 attached to the fabrication chamber 402 is embedded with the photo-stimulation source 409 and is configured to actuate the fabrication chamber 402 along x-axis direction; the translational stage 410 attached to the receptacle 406 is configured to actuate the receptacle along the x-axis direction. The arrangement of allowing longitudinal (y-axis) and vertical (z-axis) movements of the microscopic device 403, the cell sorting device 404 and the cell model dispenser 405 could enhance the sampling size and also the compatibility to a wide variety of fabrication chamber/receptacle in different types, shapes, and/or sizes. If necessary, the two translational stages (407, 410) attached to the fabrication chamber 402 and the receptacle 406 can also be configured to move along the longitudinal (y-axis) direction (not shown in FIG. 4), in addition to the lateral (x-axis) direction. Similarly, the semi-opened case 411 can also be configured to move along the lateral (x-axis) direction (not shown in FIG. 4) for facilitating image capturing process by the microscopic device 403 over the x-y planar surface of the fabrication chamber 402 and dispensation of fabricated BECMs after separation by the cell sorting device 404 through the cell model dispenser 405 into the receptacle(s) 406 along both x- and y-axis directions. In one configuration of the third embodiment of the present system, the semi-opened case 411 is configured to have at least two openings each for a part of or a whole microscopic device 403 and a part of or a whole cell model dispenser 405 to move along the vertical (z-axis) direction towards or away from the fabrication chamber 402 and the receptacle 406, respectively. The actuations of the semi-opened case 411, the microscopic device 403 and the cell model dispenser 405 along different directions are preferably controlled by the image analysis software of the present system as described herein.

Turning to FIG. 5, an example of fabrication chamber being selected from a microfluidics 500 comprising one fluid inlet for injecting a biomaterial-cell mass mixture 501, one fluid outlet 503, and one or more additional fluid inlets (502a, 502b, 502c . . . ) for injecting substances other than the biomaterial/cell masses including, but not limited to, supplements for growth and maintenances of cell masses, test compound (e.g., drug), labelling dyes, buffer etc. The main channel region 504 may be convergent into a single channel or divergent into more channels, or be divided into multiple compartments subject to the experimental design (which is not shown in FIG. 5). It should be understood that other components such as microfluidic valves, microchannels, fluids mixing chamber, filtration membrane, collection chamber and waste chamber can be incorporated into the fabrication chamber subject to the needs of the precise fabrication of the BECMs.

### Example 2—Fabrication of Colon Cancer Spheroid Model From Colorectal Adenocarcinoma Cell Line HT-29 by the Present System

Initially, spheroids of colorectal adenocarcinoma HT-29 were grown on a low-attachment culture dish and were maintained in RPMI-1640 medium supplemented with 10% fetal bovine serum and penicillin-streptomycin. All cells were grown at 37° C. in a humidified 5% CO₂ atmosphere before harvesting. Spheroids were formed from micron- to millimeter-size in their average diameter.

Gelatin-methacryloyl (GelMA) based photo-crosslinkable hydrogel was used as the precursor of the biomaterial for encapsulation in this example. In detail, the GelMA based photo-crosslinkable hydrogel such as gelatin-methacryloyl with 90 bloom and a photoinitiator such as lithium phenyl-2,4,6-trimethylbenzoylphosphinate (LAP) were used to form the biomaterial. 20% v/v of GelMA and 0.5% v/v LAP were dissolved in phosphate buffered saline (PBS) as a formulation. Before use, the formulation was pre-warmed to 37° C.

The spheroids collected from the culture dish were filtered through a mesh with specific cut-off size substantially smaller than the target spheroids but larger than other masses than the target spheroids, e.g., in a size range from 100 to 5000 μm. They were then stained with specific fluorescence probes to colon carcinoma, e.g., Calcein AM, suspended in a PBS solution to form a spheroid suspension, followed by mixing with the pre-warmed formulation in a volume ratio such that the final mixture is with at least the minimum gelation concentration of the biomaterial inside the fabrication chamber described in Example 1. In certain embodiments, the fabrication chamber contains multiple compartments, and the biomaterial formulation is loaded into each of these multiple compartments which carries fluorescence-labeled spheroids of various sizes which should be evenly spread and ready for subsequent analysis.

The microscopic device described in Example 1 was used to scan the whole fabrication chamber of cells using visible light, and lasers with wavelengths of 488 nm and 633 nm. Bright-field and fluorescent images of the whole chamber were captured. A range of light sources could be chosen, and excitation and emission filters could be used to match the excitation and emission wavelength of the labelling dye used. The light source could be selected from incandescent tungsten-halogen bulb, laser or light emitting diode (LED). The captured images by the camera of the microscopic device were imported to an image analysis software (i.e., 201 in FIG. 2) to analyze and identify target spheroid based on the following parameters of each spheroid: mass (e.g., estimated volume/cell density), size (e.g., planar arca or volume of target spheroid), position (e.g., nuclear distance between target spheroids and other spheroids or edge distance between target spheroids and debris), surface uniformity, content consistency, signal intensity (e.g., fluorescent signal intensity detected by each channel compared to control or a reference signal intensity), etc. It should be understood that the above list of parameters is not exhaustive, and the number of parameters to be analyzed by the image analysis software can vary according to the interest and needs of the user and/or selection criteria for each instance. Particular value(s) of the above parameters may be inputted as selection criteria manually or selected by the software automatically according to the interest and/or needs of the user. For instance, the spheroids ranging from 80-100 μm in diameter, with both green and red fluorescent signals inside the spheroid, and with at least 200 μm of separation between the target spheroid and other neighboring, non-target cell masses which exceeded 20 μm in diameter were selected as target spheroids in this example. The software then generated three-dimensional (3-D) coordinates of all the selected spheroids and proceeded to biomaterial encapsulation.

Light irradiation was applied to the coordinates of interest to crosslink the GelMA around the target cell masses. A gel size of 400 μm in diameter was used to enclose each of the target spheroids. Since the photo-crosslinkable hydrogel used in this example was UV- or near UV-curable, 100 seconds of light irradiation with a wavelength of 405 nm was performed to complete the cross-linking process, generating gel-encapsulated spheroids where each spheroid had a diameter of about 80-100 μm and both green and red fluorescent signals inside the spheroids (FIG. 7).

### Example 3—High-Throughput Fabrication of Cell Models Based on Spheroids or Organoids Isolated From BECMs

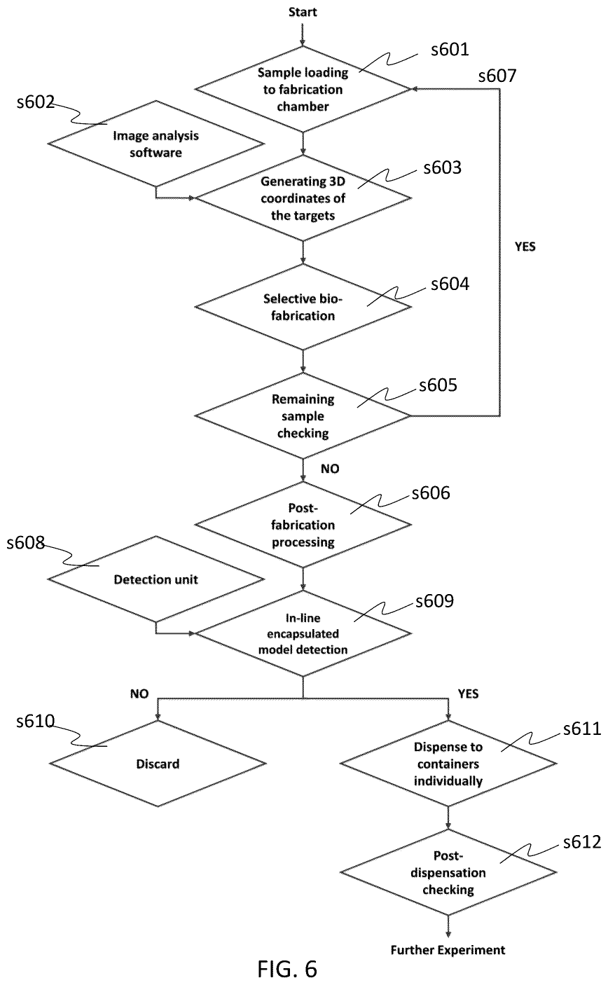

FIG. 6 summarizes the key steps of the present method from sample loading to a bio-fabrication chamber containing multiple compartments, analysis of the images of the loaded sample containing both the target and non-target cell masses in the chamber acquired by the cell imaging device, generation of 3-D coordinates of target cell masses for selective bio-fabrication by a corresponding image analysis algorithm, induction of biomaterial encapsulation after selection, and a post bio-fabrication checking and processing. For subsequent high-throughput analysis based on the as-fabricated BECMs, FIG. 6 further outlines the steps of sorting the as-fabricated BECMs according to certain requirements for said subsequent analysis including dispensation of the selected BECMs into a corresponding receptacle through the cell model dispenser described in Example 1 for high-throughput analysis and a post-dispensation checking before the analysis starts. An example of the fabricated BECM after sorting by the cell sorting device and dispensed by the cell model dispenser is depicted in FIG. 8.

Taking the microfluidic chip 500 of FIG. 5 as an illustrative example of the fabrication chamber, a pool of cell masses from a biological sample mixed with a GelMA based photo-crosslinking hydrogel and a corresponding photo-initiator was loaded to the microfluidic chip through the inlet 501 by a syringe pump (s601) until the cavity (the main channel region 504) inside the microfluidic chip 500 was filled up with the mixture. The microfluidic chip 500 was then actuated by the 3D moving mechanism to capture microscopic images. Image analysis was performed to identify and select the interested targets for bio-fabrication (s602) (i.e., selective encapsulation of target cell mass(es) by the biomaterial). 3D coordinates of the interested targeted were then generated (s603). Selective bio-fabrication utilized the coordinates generated by the image analysis software to encapsulate the targets (s604). In this example, multiple 405-nm light beams with a diameter of 400 μm were used to irradiate all the coordinates of interest synchronously. Multiple encapsulated models were formed as a result. After the selective bio-fabrication, warm phosphate-buffered saline (PBS) solution was pumped to flush the encapsulated models and unsolidified biomaterial out of the microfluidic chip through the outlet 503 to the cell sorting device. Thus, one cycle of selective bio-fabrication was completed.

In the case where sealed glassware or plastic containers were used instead, no syringe pump or PBS buffer flushing were required. Image analysis and selective bio-fabrication would be performed once in the whole process.

The syringe pump could be provided with a sensor to check the content of the syringe to determine the operation of the next cycle (s605). If all the mixture inside the syringe was pumped out, post-fabrication process would be carried out (s606). On the contrary, the next cycle of bio-fabrication would be performed (s607).

The fabricated biomaterial-encapsulated cell masses from the microfluidic chip were sorted by a detection unit (s608) in the cell sorting device, an example of which is depicted in FIG. 2c, in which the detector included at least a light emitter and a detector. If certain selection criteria were met, e.g., size, mass, surface uniformity, content consistency, positive fluorescent signal(s), etc., that or those fabricated biomaterial-encapsulated cell masses would be selected (s609) transferred to a connected dispenser. Otherwise, the other non-target cell masses or cell debris would be discarded (s610). The selected, fabricated biomaterial-encapsulated cell masses would be dispensed to each of the specimen containers in a high throughput manner, e.g., to a 96-well or 384-well plate as shown in FIG. 2d (s611). After dispensing the selected, fabricated biomaterial-encapsulated cell masses to the corresponding specimen container, a post-dispensation check was performed under a spectrophotometer or microscope (s612). FIG. 8 shows an example of post-dispensation checking by fluorescent imaging. Each of the selected, fabricated biomaterial-encapsulated cell masses was checked by fluorescent microscopy to ensure correct cell phenotype(s) was/were encapsulated and sorted.

Although the invention has been described in terms of certain embodiments, other embodiments apparent to those of ordinary skill in the art are also within the scope of this invention. Accordingly, the scope of the invention is intended to be defined only by the claims which follow.

### INDUSTRIAL APPLICABILITY

Configuration of the present system enables high throughput selection, fabrication and separation of biomaterial-encapsulated cell mass model from a pool of cell mass models. The present system can also be configured into a continuous liquid handling system from cell mass selection, biomaterial-encapsulated cell mass fabrication, to sorting target cell mass model in a sterilized setting such that sample contamination by external factors can be minimized. The as-fabricated biomaterial-encapsulated cell mass model or biological cells derived therefrom are useful in different areas including drug screening, pharmacokinetic study, drug resistance study, cancer staging, metastasis study, as a xenograft in in vivo models, as an implant for regenerative medicine, or selection of certain cell phenotypes from a sample, tissue or biopsy, immunotherapy, and a platform for studying molecular pathway, stem cell research, or developmental biology. The present system is also easy to be assembled and adjustable (including scale up or down) according to the interest or needs of the user and selection criteria, for each instance.

What is claimed is:

1. A system comprises:
a fabrication chamber comprising at least one fluid inlet, one fluid outlet, and a main channel region;
a multi-axial movable mechanism disposed adjacent to, above or under the fabrication chamber;
a photo-stimulation unit disposed above or under the fabrication chamber;
a microscopic device disposed at an opposing side of the photo-stimulation unit;
a cell sorting device comprising at least one fluid inlet communicating with the fluid outlet of the fabrication chamber, a target cell masses detection unit, and one fluid outlet;
a cell model dispenser comprising one end communicating with the fluid outlet of the cell sorting device, and one or more dispenser heads disposed on the other end; and
a receptable.

2. The system of claim 1, wherein the fabrication chamber is a microfluidics, a single-channel device, or a multi-channel device.

3. The system of claim 1, wherein the fabrication chamber is optically transparent.

4. The system of claim 1, wherein the main channel region of the fabrication chamber comprises multiple compartments.

5. The system of claim 1, wherein the microscopic device comprises a microscope and a camera.

6. The system of claim 5, wherein the microscope comprises a light source comprising incandescent tungsten-halogen bulb, laser or light emitting diode (LED).

7. The system of claim 5, wherein the microscope has an adjustable depth of focus.

8. The system of claim 4, further comprising an image analysis software for processing and analyzing images captured by the microscopic device.

9. The system of claim 8, wherein the image analysis software is configured to identify region of interests (ROIs) in the images, map the cell masses of interest in the ROIs with the corresponding location in the fabrication chamber, in order to determine three-dimensional (3-D) coordinates of the target cell masses in the fabrication chamber.

10. The system of claim 9, wherein the image analysis software is also configured to differentiate the optical signals from the target cell masses than those from the non-target cell masses or any noise from the background, and calculate a distance between the target cell masses and neighboring non-target cell masses thereof.

11. The system of claim 1, wherein the photo-stimulation unit comprises at least one light source with various wavelengths and a plurality of filters.

12. The system of claim 1, wherein the multi-axial movable mechanism is a three-dimensional (3-D) translational stage configured to move along at least two axes perpendicular to the light source direction of the microscopic device, respectively.

13. The system of claim 12, wherein the 3-D translational stage is disposed under the fabrication chamber.

14. The system of claim 12, wherein the photo-stimulation unit is disposed under the fabrication chamber and within the 3-D translational stage.

15. The system of claim 1, wherein the multi-axial movable mechanism is a movable clamping structure configured to move the fabrication chamber along at least two axes perpendicular to the light source direction of the microscopic device, respectively

16. The system of claim 15, wherein the photo-stimulation unit is disposed under or above the fabrication chamber.

17. The system of claim 4, wherein one or more compartments of the main channel region of the fabrication chamber are masked by a plurality of filters of the photo-stimulation unit such that the light source of the photo-stimulation unit only reaches the unmasked compartments.

18. The system of claim 8, wherein the image analysis software is also configured to control the movement of the multi-axial movable mechanism according to the 3-D coordinates of the target cell masses in the fabrication chamber and align with a light path of a light source of the photo-stimulation unit.

19. The system of claim 1, wherein the target cell masses detection unit of the cell sorting device comprises a quenching mechanism comprising a light scattering device.

20. The system of claim 1, wherein the cell sorting device and the cell model dispenser are configured into a single unit.

21. The system of claim 1, wherein the cell sorting device and the cell model dispenser are two separate units.

22. The system of claim 1, 20, or 21, wherein the cell model dispenser further comprises a detection unit comprising ultrasound object detector, infrared detector photomultiplier tubes (PMT), photodiodes and excitation light sources.

23. The system of claim 1, wherein the receptacle comprises multi-well plates, petri dishes, centrifuge tubes or other common labware configured to receive and carry separated BECMs from the cell model dispenser.

24. A method for fabricating biomaterial-encapsulated cell mass (BECM) model comprising using the system of any one of the preceding claims, said using the system comprising:
providing a pool of cell masses;
pre-labelling the cell masses with one or more fluorescent or luminescent probes specific to one or more biomarkers expressed in target cell masses;
mixing the pre-labelled cell masses with a biomaterial formulation to form a first mixture, wherein the biomaterial formulation comprises a precursor of the biomaterial;
capturing a first batch of images of the fabrication chamber for constructing a three-dimensional geometry of the fabrication chamber;
providing the first mixture to the fabrication chamber;
capturing a second batch of images of the fabrication chamber provided with the first mixture to detect any fluorescent or luminescent signals;
determining region of interests (ROIs) from the second batch of images;
identifying centroids of the cell masses from the ROIs meeting a set of threshold values with the presence of fluorescent or luminescent signals;
determining three-dimensional (3-D) coordinates of the target cell masses in the fabrication chamber by an image analysis software;
selectively encapsulating the target cell masses into the biomaterial according to the determined three-dimensional coordinates of the target cell masses in the fabrication chamber in order to form a second mixture comprising the BECMs;
transferring the second mixture to the cell sorting device for separation of the BECMs from non-target cell masses or cell debris; and
dispensing the separated BECMs to the receptacle through the cell model dispenser.

25. The method of claim 24, wherein the pool of cell masses is sourced from primary, secondary or modified cell lines, or a tissue or biopsy of the same or different subject than a recipient of the BECM or the cells derived therefrom.

26. The method of claim 24, wherein the cell masses comprise spheroids and organoids.

27. The method of claim 24, wherein the first mixture is provided through the fluid inlet to the fabrication chamber.

28. The method of claim 27, wherein the precursor of the biomaterial is a photo-crosslinkable hydrogel.

29. The method of claim 28, wherein the biomaterial formulation further comprises a photo-initiator.

30. The method of claim 28, wherein the photo-crosslinkable hydrogel in the presence of the photo-initiator is cross-linked under a selective light irradiation by a photo-stimulation unit to the fabrication chamber at where the 3-D coordinates of the target cell masses are determined.

31. The method of claim 24, wherein the photo-stimulation unit comprises at least one light source and a plurality of filters.

32. The method of claim 31, wherein the plurality of filters masks one or more of the compartments of the main channel region of the fabrication chamber such that the light source of the photo-stimulation unit only reaches the unmasked compartments.

33. The method of claim 32, wherein masking of the one or more compartments by the plurality of filters is controlled by the image analysis software according to the 3-D coordinates of the target cell masses.

34. The method of claim 24, wherein the multi-axial movable mechanism attached to the fabrication chamber for actuating the fabrication chamber along at least two axes that are perpendicular to the light source direction of the microscopic device, respectively is also controlled by the image analysis software.

35. The method of claim 34, wherein the photo-stimulation unit is disposed under the fabrication chamber.

36. The method of claim 24, wherein the first and second batches of images are captured by the microscopic device comprising a microscope and a camera.

37. The method of claim 24, wherein the microscopic device is configured to detect optical signals including visible light, fluorescent, luminescent, scattering, absorbance, and turbidity signals and output bright-field, dark-field, fluorescent, and luminescent images.

38. The method of claim 24, wherein said constructing the three-dimensional geometry of the fabrication chamber from the first batch of images is also performed by the image analysis software.

39. The method of claim 38, wherein the image analysis software is also configured to differentiate the optical signals from the target cell masses than those from the non-target cell masses or any noise from the background, and also calculate distance between the target cell masses and their neighboring non-target cell masses.

40. The method of claim 24, wherein the separation of the BECMs from the non-target cell masses or cell debris in the cell sorting device is performed by the target cell masses detection unit, and the target cell masses detection unit is controlled either by the image analysis software or by a master software that allows the target cell masses detection unit to automatically identify the BECMs based on a set of selection criteria and separate them from the non-target cell masses or cell debris in the second mixture.

41. The method of claim 40, wherein the set of selection criteria comprises one or more of average diameter of the target cell masses, average diameter of non-target cell masses, expression of one or more biomarkers, composition of BECMs, surface uniformity, content consistency, and fluorescence intensity upon quenching by the cell sorting device.

42. The method of claim 24, further comprising removing any remaining fluid containing non-target cell masses, cell debris and other materials from the fabrication chamber, cell sorting device, and cell model dispenser by one or more rounds of buffer washing prior to providing a subsequent mixture of the cell masses and the biomaterial formulation to the fabrication chamber of the system.
